(19) United States
(12) Patent Application Publication
(10) Pub. No.: US 2018/0110958 A1
(43) Pub. Date: Apr. 26, 2018

(54) SLEEPING ENVIRONMENT CONTROL SYSTEM AND METHOD

(71) Applicant: Industrial Technology Research Institute, Hsinchu (TW)

(72) Inventors: Hsiao-Yuan Wu, Hsinchu City (TW); Nien-Tzu Chao, Taoyuan City (TW); Chih-Jian Hu, Hsinchu City (TW)

(73) Assignee: Industrial Technology Research Institute, Hsinchu (TW)

(21) Appl. No.: 15/394,780

(22) Filed: Dec. 29, 2016

(30) Foreign Application Priority Data
Oct. 21, 2016 (TW) 105134041

(51) Int. Cl. A61M 21/02

(52) U.S. Cl. CPC A61M 21/02; A61M 2021/0066

(57) ABSTRACT

A sleeping environment control system and method includes sensing a condition of environment to obtain a value of an environmental condition parameter, and then generating a value of a thermal sensation indicator according to the value of the environmental condition parameter. A physiological status of a user is sensed to obtain a value of a physiological status parameter. After a plurality of values of the thermal sensation indicator and a plurality of values of the physiological status parameter are collected, a regression analysis is performed to obtain a best value of the thermal sensation indicator according to the plurality of values of the thermal sensation indicator and the plurality of values of the physiological status parameter. A value of an environmental condition setting parameter is adjusted according to the best value of the thermal sensation indicator.

### CROSS-REFERENCE TO RELATED APPLICATION

The present application is based on, and claims priority from, Taiwan Application Serial Number 105134041, filed on Oct. 21, 2016, the disclosure of which is hereby incorporated by reference herein in its entirety.

### BACKGROUND

### 1. Technical Field

The present disclosure generally relates to a sleeping environment control system and method, in particular, to a sleeping environment control system and method which can control the sleeping environment according to comfort parameters of user.

### 2. Related Art

In general, almost everyone has an experience about cold wake-up or warm wake-up. As indicated by research, for Hong Kong in a sub-tropical climate with reinforced concrete building structures, 60% of air-conditioning users say that they ever experience cold wake-up during sleep. According to the research, the thermal comfort condition is not just related to the air temperature but also other factors, such as humidity, wind speed, radiant temperature, human metabolic rate, fabric thermal resistance and so on. However, the comfortable-sleep function embedded in the air-conditioner can only control the air temperature according to a pre-set curve, but cannot also consider the other factors. As a result, it cannot satisfy the requirement for the comfort condition.

On the other hand, the phenomena of cold wake-up and warm wake-up indicate that human bodies do react to the thermal condition of environment during sleeping. However, it is still an issue to be clarified about how to define or measure the thermal comfort condition during sleeping. Because of inability to know the immediate subjective feelings of the person during sleeping, it would be very helpful for establishing a sleeping environment control system and method if measurable objective parameters can be used as sleep comfort indicators.

### SUMMARY

The disclosure provides a sleeping environment control system and method thereof.

In an embodiment, the disclosure provides a sleeping environment control system, comprising a thermal sensation indicator module, a physiological status module, an analysis module, and a control module. The thermal sensation indicator module is adapted to sense a condition of environment to obtain a value of an environmental condition parameter, and generate a value of a thermal sensation indicator according to the value of the environmental condition parameter. The physiological status module is adapted to sense a physiological status of a user to obtain a value of a physiological status parameter. The analysis module comprises a storage unit and a calculating unit, wherein the storage unit receives and stores the value of the thermal sensation indicator from the thermal sensation indicator module and the value of the physiological status parameter from the physiological status module, after a plurality of values of the thermal sensation indicator and a plurality of values of the physiological status parameter are collected, the calculating unit performs a regression analysis to obtain a best value of the thermal sensation indicator according to the plurality of values of the thermal sensation indicator and the plurality of values of the physiological status parameter stored in the storage unit. The control module receives the best value of the thermal sensation indicator and adjusts a value of an environmental condition setting parameter according to the best value of the thermal sensation indicator.

In another embodiment, the disclosure provides a sleeping environment control method, comprising a step of sensing a condition of environment to obtain a value of an environmental condition parameter, and generating a value of a thermal sensation indicator according to the value of the environmental condition parameter. Further, a physiological status of a user is sensed to obtain a value of a physiological status parameter. After a plurality of values of the thermal sensation indicator and a plurality of values of the physiological status parameter are collected, a regression analysis is performed to obtain a best value of the thermal sensation indicator according to the plurality of values of the thermal sensation indicator and the plurality of values of the physiological status parameter. A value of an environmental condition setting parameter is adjusted according to the best value of the thermal sensation indicator.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 schematically illustrates a block structure of sleeping environment control system, according to an exemplary embodiment of the disclosure.

FIG. 2 is a flow diagram of sleeping environment control method, according to an exemplary embodiment of the disclosure.

FIG. 3 schematically illustrates a relation between the RR parameter of heart rate variability and the sleeping time, according to an exemplary embodiment of the disclosure.

FIG. 4 schematically illustrates a relation between the Delta wave intensity of brain waves and the sleeping time, according to an exemplary embodiment of the disclosure.

FIG. 5 schematically illustrates regression function curves from a regression analysis based on the physiological status parameter (Ps) and the thermal sensation indicator (I), according to an exemplary embodiment of the disclosure.

FIG. 6 schematically illustrates a regression function curve from a regression analysis based on the physiological status parameter (Ps) and the thermal sensation indicator (I), according to another exemplary embodiment of the disclosure.

FIG. 7 is a flow diagram of sleeping environment control method, according to another exemplary embodiment of the disclosure.

### DESCRIPTION OF THE EMBODIMENTS

Reference will now be made in detail to the present preferred embodiments of the disclosure, examples of which are illustrated in the accompanying drawings. Wherever possible, the same reference numbers are used in the drawings and the description to refer to the same or like parts.

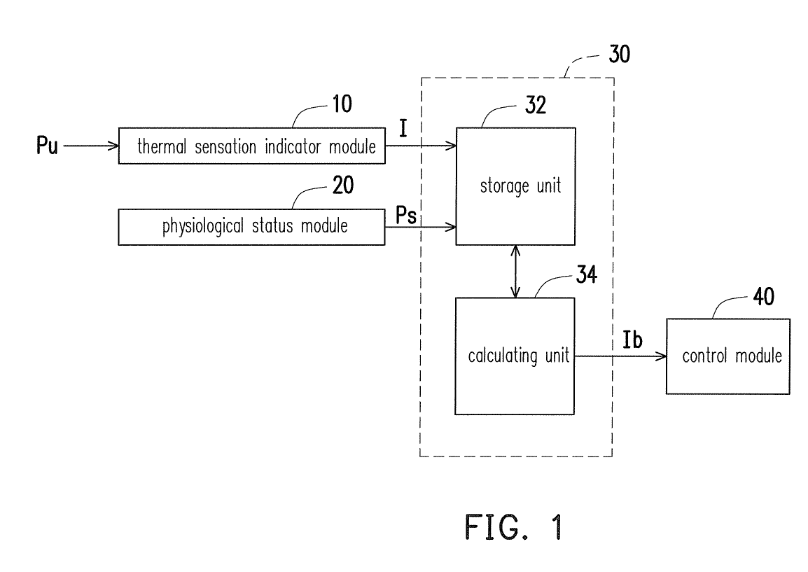

FIG. 1 schematically illustrates a block structure of sleeping environment control system, according to an exemplary embodiment of the disclosure. Referring to FIG. 1, the disclosure provides a sleeping environment control system 1, which includes a thermal sensation indicator module 10, a physiological status module, 20, an analysis module 30 and a control module 40. The thermal sensation indicator module 10 is adapted to sense a condition of environment to obtain a value of an environmental condition parameter (Pe), and generate a value of a thermal sensation indicator (I) according to the value of the environmental condition parameter (Pe). The physiological status module 20 is adapted to sense a physiological status of a user to obtain a value of a physiological status parameter (Ps). The analysis module 30 comprises a storage unit 32 and a calculating unit 34. The storage unit 32 receives and stores the value of the thermal sensation indicator (I) from the thermal sensation indicator module 10 and the value of the physiological status parameter (Ps) from the physiological status module 20. After a plurality of values of the thermal sensation indicator (I) and a plurality of values of the physiological status parameter (Ps) are collected, the calculating unit 34 performs a regression analysis to obtain a best value of the thermal sensation indicator (Ib) according to the plurality of values of the thermal sensation indicator (I) and the plurality of values of the physiological status parameter (Ps) stored in the storage unit 32. The control module 40 receives the best value of the thermal sensation indicator (Ib) and adjusts a value of an environmental condition setting parameter (Pes) according to the best value of the thermal sensation indicator (Ib).

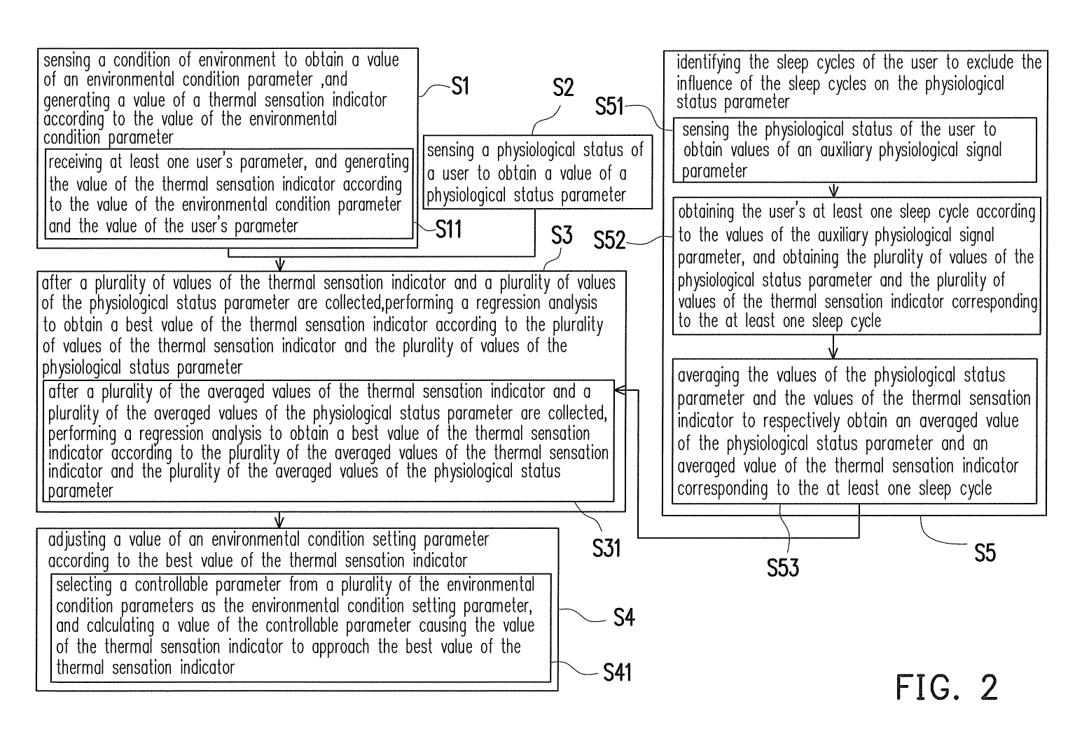

FIG. 2 is a flow diagram of the sleeping environment control method, according to an exemplary embodiment of the disclosure. Referring to FIG. 1 and FIG. 2, the sleeping environment control method of the disclosure includes the steps of:

Step S1: sensing a condition of environment to obtain a value of an environmental condition parameter (Pe), and generating a value of a thermal sensation indicator (I) according to the value of the environmental condition parameter (Pe).

Step S2: sensing a physiological status of a user to obtain a value of a physiological status parameter (Ps).

Step S3: after a plurality of values of the thermal sensation indicator (I) and a plurality of values of the physiological status parameter (Ps) are collected, performing a regression analysis to obtain a best value of the thermal sensation indicator (Ib) according to the plurality of values of the thermal sensation indicator (I) and the plurality of values of the physiological status parameter (Ps).

Step S4: adjusting a value of an environmental condition setting parameter (Pes) according to the best value of the thermal sensation indicator (Ib).

For detail, in the step S1, the thermal sensation indicator module 10 can sense the condition of environment in use, so as to obtain the value of the environmental condition parameter (Pe) and generate the value of the thermal sensation indicator (I) according to the value of the environmental condition parameter (Pe). The environmental condition parameter (Pe) can include at least one of air temperature, relative humidity, wind speed, and mean radiant temperature. The thermal sensation indicator (I) can be one of a comfort index of Pierce two-node model, a TSV index of KSU two-node model, an index of predicted mean vote (PMV), operative temperature and air temperature. In addition, the step S1 for generating the value of the thermal sensation indicator (I) can further include a step S11: the thermal sensation indicator module 10 receives at least one user's parameter (Pu), and generates the value of the thermal sensation indicator (I) according to the value of the environmental condition parameter (Pe) and the value of the user's parameter (Pu). Wherein, the user's parameter (Pu) can include at least one of the user's fabric thermal resistance and human metabolic rate.

In an exemplary embodiment, the thermal sensation indicator (I) can take the index of predicted mean vote (PMV). According to the definition, the index of predicted mean vote (PMV) is a function of six parameters of air temperature, relative humidity, wind speed, mean radiant temperature, user's fabric thermal resistance of clothing or bedding, and human metabolic rate. In the exemplary embodiment, the thermal sensation indicator module 10 senses the condition of environment in use to respectively generate values of several environmental condition parameters (Pe) including air temperature, relative humidity, wind speed, mean radiant temperature, and so on. The thermal sensation indicator module 10 also receives values of several user's parameters (Pu), respectively representing the user's fabric thermal resistance of clothing or bedding and the human metabolic rate. The thermal sensation indicator module 10 then generates the value of the thermal sensation indicator (I) according to the values of the environmental condition parameters (Pe) and the values of the user's parameters (Pu). In addition, the fabric thermal resistance can be set by referring to the content in the publication by “Lin, Zhongping” and “Shiming Deng”, “A study on the thermal comfort in sleeping environments in the subtropics—Measuring the total insulation values for the bedding systems commonly used in the subtropics.” Building and Environment 43.5 (2008): 905-916. The human metabolic rate can be obtained according to at least one of the user's sex, age, height, and weight, a basal metabolic rate formula and a sleep metabolic rate curve (Please refer to a publication of Katayose, Yasuko, et al.: “Metabolic rate and fuel utilization during sleep assessed by whole-body indirect calorimetry.” Metabolism 58.7 (2009): 920-926.”). It is noted that the calculation above is just for providing an available manner to perform but is not the limitation.

The thermal sensation indicator (I) represents the thermal sensation of most of the users caused by the condition of environment in use. Although the comfort level of the environment condition for each one may be different, the trend is basically the same. In other words, when the environment changes and the subjective feeling becomes hotter, the thermal sensation indicator (I) reflects the trend of turning hotter.

In step S2, the physiological status module 20 senses the physiological status of a user to obtain a value of a physiological status parameter (Ps). According to the medical research, the sympathetic and parasympathetic activity intensities of the autonomic nervous system are related to the level of human comfort. The sympathetic intensity is weaker in a more comfortable state while the parasympathetic intensity is stronger. This trend also exists during sleeping. Thus, the physiological status parameter (Ps) basically needs to reflect the sympathetic or parasympathetic activity intensity. The brain waves or heart rate variability parameters are suitable for the application. In an exemplary embodiment, the physiological status parameter (Ps) can be one of RR parameter, total power (TP), high frequency power (HF), low/high frequency power ratio (LF/HF) of heart rate variability (HRV), Alpha (α) wave intensity, Beta (β) wave intensity, and Delta (δ) wave intensity of brain waves. In detail, the Beta (β) wave intensity is positively proportional to the sympathetic intensity. The high frequency power of heart rate variability is positively proportional to the parasympathetic intensity. The low/high frequency power ratio is positively proportional to sympathetic intensity.

In step S1 and step S2, a sampling time cycle can be set to perform fetching parameter data. For example, sensing and calculation can be done once in every 15 minutes, so as to generate the value of the thermal sensation indicator (I) and the value of physiological status parameter (Ps) and transmit the values to the storage unit 32 of the analysis module 30 for storage. In step S3, after a plurality of sampling data are collected, the calculating unit 34 fetches the values of the thermal sensation indicator (I) and the values of the physiological status parameter (Ps) stored in the storage unit 32 to perform a regression analysis to calculate a best value of the thermal sensation indicator (Ib).

Sleep has cyclical changes, changing from rapid eye motion (REM) to nonrapid eye motion (NREM) and returning to the REM. The intensities of sympathetic activities and parasympathetic activities change with the sleep cycle T. The physiological status parameters (Ps), such as brain waves and heart rate variability parameters, also change with the sleep cycle T. Thus, to have more precise analysis, it is necessary to exclude the influence of sleep cycles on the physiological status parameter (Ps). In an exemplary embodiment, the sleeping environment control method as provided in the disclosure further includes step S5: identifying the sleep cycles of the user to exclude the influence of the sleep cycles on the physiological status parameter (Ps). In detail, the step S5 includes:

Step S51: sensing the physiological status of the user to obtain values of an auxiliary physiological signal parameter (Pa).

Step S52: obtaining the user's at least one sleep cycle (T) according to the values of the auxiliary physiological signal parameter (Pa), and obtaining the plurality of values of the physiological status parameter (Ps) and the plurality of values of the thermal sensation indicator (I) corresponding to the at least one sleep cycle.

Step S53: averaging the values of the physiological status parameter (Ps) and the values of the thermal sensation indicator (I) to respectively obtain an averaged value of the physiological status parameter (Ps) and an averaged value of the thermal sensation indicator (I) corresponding to the at least one sleep cycle (T).

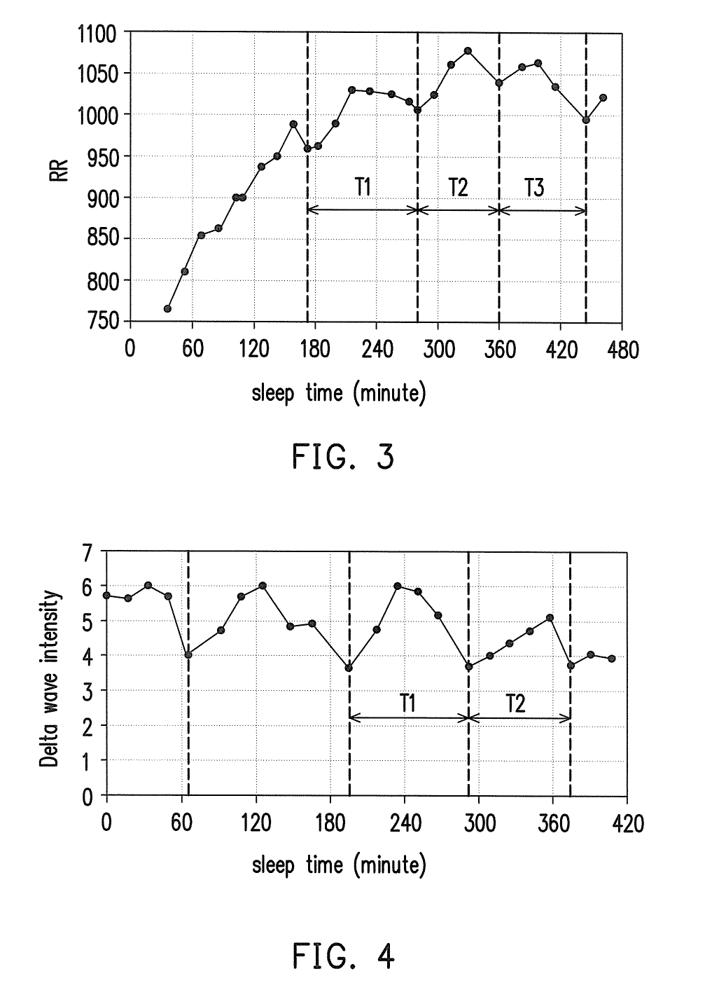

In step S51, the auxiliary physiological signal parameter (Pa) can be obtained by the physiological status module 20. In step S52, the auxiliary physiological signal parameter (Pa) can be transmitted to the analysis module 30, and the analysis module 30 obtains the user's at least one sleep cycle (T) according to values of the auxiliary physiological signal parameter (Pa). Then the analysis module 30 obtains the values of the physiological status parameter (Ps) and the values of the thermal sensation indicator (I) corresponding to the at least one sleep cycle (T) from the storage unit 32. Wherein, the auxiliary physiological signal parameter (Pa) can be one of the RR parameter of the HRV, the Alpha wave intensity and Delta wave intensity. In an exemplary embodiment, the auxiliary physiological signal parameter (Pa) is the RR parameter of the HRV. FIG. 3 schematically illustrates a relation between the RR parameter of heart rate variability and the sleeping time, according to an exemplary embodiment of the disclosure. The value of the RR parameter in the transition period between two sleep cycles reaches to a local minimum. Base on this, time periods between two adjacent minimums are observed. In general, a sleep cycle T is about 60 to 120 minutes long. If the length of a time period between two minimums is within this range, the time period can be reasonably considered as a sleep cycle. Otherwise, the data within the time period is removed. Take FIG. 3 as an example: the sleep cycles are T1, T2, and T3 respectively, based on analysis on the RR parameter. In another exemplary embodiment, the auxiliary physiological signal parameter (Pa) can be the Delta wave intensity of brain waves. FIG. 4 schematically illustrates a relation between the Delta wave intensity of brain waves and the sleeping time, according to an exemplary embodiment of the disclosure. As seen in FIG. 4, the Delta wave intensity of brain waves behaves similarly to the RR parameter of HRV and can be used to determine the sleep cycles T. Then the analysis module 30 further finds the values of the physiological status parameter (Ps) and the values of the thermal sensation indicator (I) from the storage unit 32 corresponding to each sleep cycle. In step S53, the analysis module 30 averages the values of the physiological status parameter (Ps) and the values of the thermal sensation indicator (I) corresponding to a sleep cycle T, so as to respectively obtain an averaged value of the physiological status parameter (Ps) and an averaged value of the thermal sensation indicator (I) of the sleep cycle T.

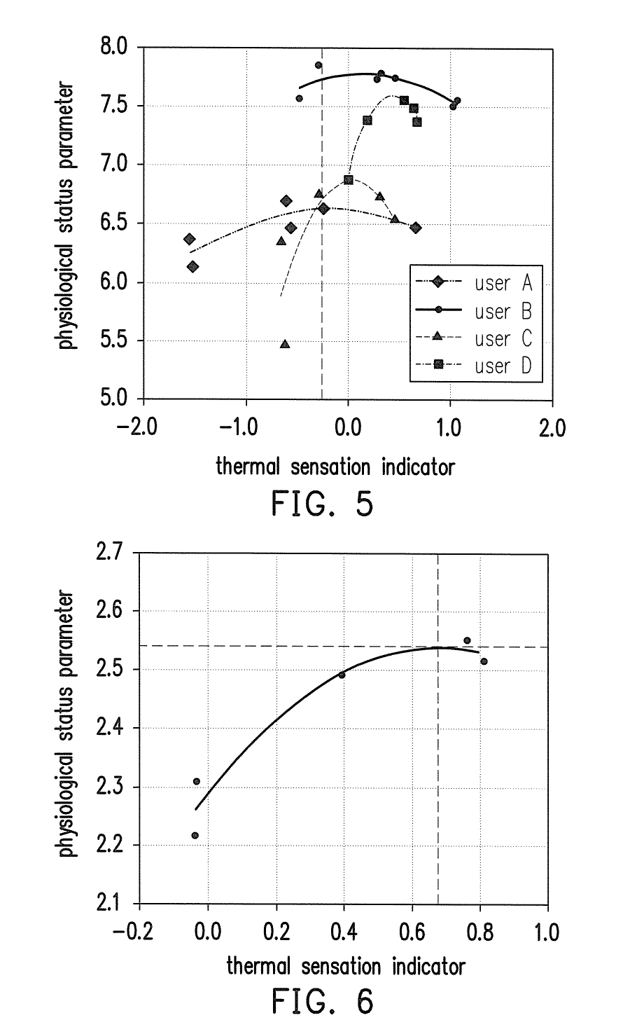

In step S3, a regression analysis is performed. After a plurality of values of the thermal sensation indicator (I) and a plurality of values of the physiological status parameter (Ps) are collected, a regression analysis is performed to calculate a best value of the thermal sensation indicator (Ib). Taking into account the sleep cycles, the regressing analysis in step S3 further includes step S31: after a plurality of the averaged values of the thermal sensation indicator (I) and a plurality of the averaged values of the physiological status parameter (Ps) are collected, performing a regression analysis to obtain a best value of the thermal sensation indicator (Ib) according to the plurality of the averaged values of the thermal sensation indicator (I) and the plurality of the averaged values of the physiological status parameter. FIG. 5 schematically illustrates regression function curves from a regression analysis based on the physiological status parameter (Ps) and the thermal sensation indicator (I), according to an exemplary embodiment of the disclosure. In the exemplary embodiment of FIG. 5, the thermal sensation indicator (I) is an index of predicted mean vote (PMV). The regression analysis uses a quadratic function as the regression function. FIG. 5 shows the results of four different users, and the data are collected during four nights of sleep for each user. The influence of sleep cycles on the physiological status parameter (Ps) is excluded using step S5. Each point in FIG. 5 represents an averaged value of the physiological status parameter (Ps) and an averaged value of the thermal sensation indicator (I) corresponding to a single sleep cycle T. Wherein, when the value of the thermal sensation indicator (I) is relatively large, it indicates a relatively hot condition. When the value of the thermal sensation indicator (I) is relatively small, it indicates a relatively cold condition. In the exemplary embodiment of FIG. 5, the HF parameter of HRV, which is positively proportional to the parasympathetic intensity, is taken as the physiological status parameter (Ps). Therefore, when the value of the physiological status parameter (Ps) is higher, it indicates that the user is at a more comfortable state. When the value of the physiological status parameter (Ps) is smaller, it indicates that the user is at a less comfortable state. Thus, the best value of the thermal sensation indicator (Ib) corresponds to the maximum point of the regression function obtained from the regression analysis. FIG. 6 schematically illustrates a regression function curve from a regression analysis based on the physiological status parameter (Ps) and the thermal sensation indicator (I), according to another exemplary embodiment of the disclosure. In the exemplary embodiment of FIG. 6, the Alpha wave intensity of the brain waves, which is positively proportional to the parasympathetic intensity, is taken as the physiological status parameter (Ps). The TSENS index in the Pierce two-node model is taken as the thermal sensation indicator (I). Although the physiological status parameter (Ps) and the thermal sensation indicator (I) used in the embodiment of FIG. 6 are different from those of the embodiment of FIG. 5, the interpretations concerning the comfort level and the thermal perception are similar. A result of user E is shown in FIG. 6. The maximum point of the quadratic regression function locates approximately at I=0.7. This indicates that the best value of the thermal sensation indicator (Ib) of the user E is 0.7. In addition, for other exemplary embodiments, the physiological status parameter (Ps) can be a parameter that is positively proportional to the sympathetic intensity. In this case, the best value of the thermal sensation indicator (Ib) corresponds to the minimum point of the regression function.

To obtain the best value of the thermal sensation indicator (Ib) with better precision, the step S3 for calculating the best value of the thermal sensation indicator (Ib) can further include: The regression analysis is performed only if the number of data points is greater than a pre-set threshold value. In an exemplary embodiment, only when the storage unit 32 has stored at least 5 sets of values of the physiological status parameter (Ps) and the thermal sensation indicator (I), the calculating unit 34 performs the regression analysis to calculate the best value of the thermal sensation indicator (Ib). However, the minimum number for sampling is not limited to this. Further, the step S3 for calculating the best value of the thermal sensation indicator (Ib) can further include: calculating the best value of the thermal sensation indicator (Ib) only when a correlation coefficient of the regression analysis is greater than or equal to a threshold value, and keeping on collecting the values of the physiological status parameter (Ps) and the values of the thermal sensation indicator (I) when the correlation coefficient of the regression analysis is less than the threshold value. In an exemplary embodiment, the threshold value for the correlation coefficient is set to 0.8.

In addition, as shown in FIG. 5, the most comfortable condition (the best value of the thermal sensation indicator (Ib)) is different for each user. And then the step S4 is performed: the control module 40 adjusts a value of an environmental condition setting parameter (Pes) according to the best value of the thermal sensation indicator (Ib) as received by the control module 40. In detail, the value of the thermal sensation indicator (I) is generated from a plurality of environmental condition parameters (Pe). The step S4 of adjusting the environmental condition setting parameter (Pes) can further include the step S41: selecting a controllable parameter from a plurality of the environmental condition parameters (Pe) as the environmental condition setting parameter (Pes), and calculating a value of the controllable parameter causing the value of the thermal sensation indicator to approach the best value of the thermal sensation indicator (Ib).

Experimental examples according to the sleeping environment control method of the disclosure are described as follows.

### Experimental Example 1

Please refer to FIG. 2 and FIG. 5, the PMV index is used as the thermal sensation indicator (I). PMV is a function of six parameters of air temperature, relative humidity, wind speed, mean radiant temperature, user's fabric thermal resistance of clothing or bedding, and human metabolic rate. The fabric thermal resistance and human metabolic rate are taken as the user's parameters (Pu) and the air temperature, relative humidity, wind speed, and mean radiant temperature are takes as the environmental condition parameters (Pe). The fabric worn by user A is short-sleeve and short-pant, the bed has mattress with mat, the quilt is thin, and the coverage rate of the body of user A by the bed and the quilt is about 60%. The total fabric thermal resistance is 0.282° C.·m²/W according to a fabric thermal resistance table. According to the sleeping time and the basal metabolic rate data, the human metabolic rate of user A is about 38 watt/m² at this time. The sampling interval is set to be 15 minutes. The relative humidity in the room is 55%. The mean radiant temperature is 27° C. Without a fan inside the room, the wind speed is assumed to be 0.1 m/s, which is caused by the natural convection and the air-conditioning airflow. The air temperature in the environment is a controllable parameter. According to the foregoing data, the best value of the thermal sensation indicator (Ib) for user A is −0.2. And when the air temperature (integer degrees) is set to 25° C., the value of the thermal sensation indicator (I) is closest to the best value of the thermal sensation indicator (Ib) for user A. Therefore the control module 40 transmits a control signal to the air conditioner to set the air temperature to 25° C. and then wait for the next sampling time. The above action is repeated until the sleeping environment control system 1 is shut down.

### Experimental Example 2

Continuing from the experimental example 1, the environment in use is assumed to have a fan. The fan has only two control states: ON or OFF. When the fan is turned on, the averaged wind speed on body surface of the user A is estimated as 0.2 m/s. When the fan is turned off, the averaged wind speed on body surface of the user A is assumed to be 0.1 m/s. In this case, the controllable parameters among the environmental condition parameters (Pe) are the wind speed and the air temperature. In this situation, the control module 40 would evaluate every possible combination of the controllable parameters in order to find the best parameter combination whose value of the thermal sensation indicator is closest to the best value of the thermal sensation indicator. For example, assume the best combination is [air temperature 26° C., fan on], and then the control signals according to the above combination are transmitted to the air conditioner and the fan.

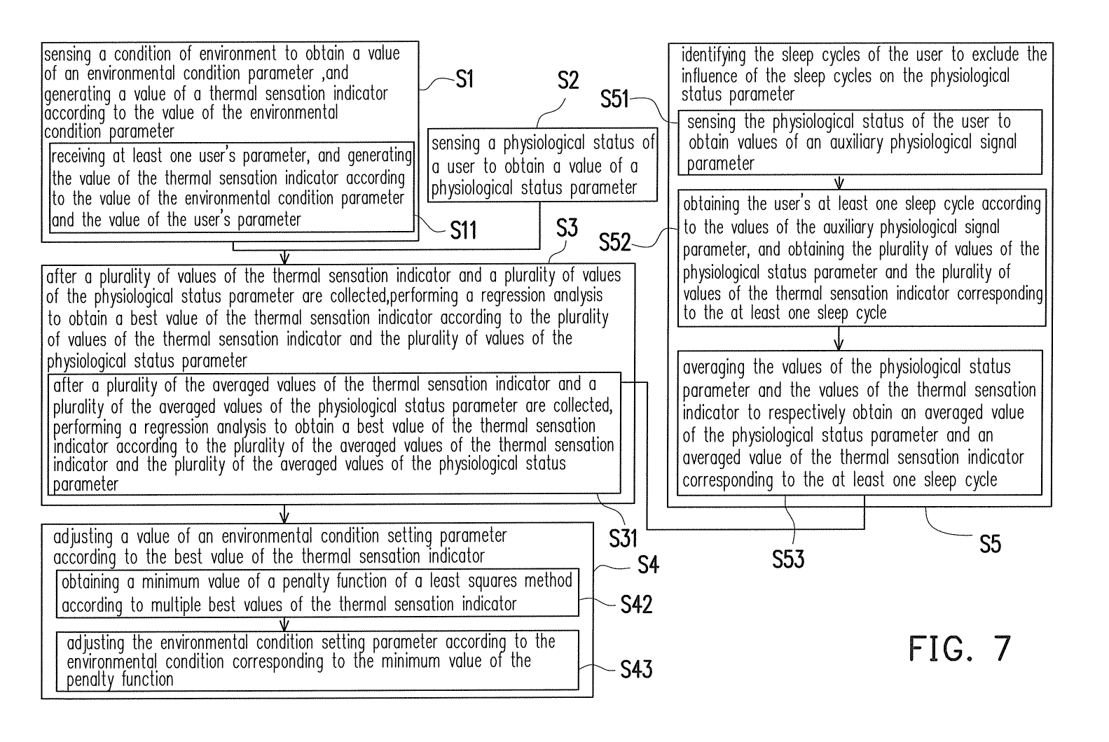

The sleeping environment control method of the disclosure can be applied to an environment with a plurality of users. Please refer to FIG. 7. Step S4 further includes step S42: obtaining a minimum value (Pₘᵢₙ) of a penalty function of a least squares method according to a plurality of best values of the thermal sensation indicator (Ib). Then step S43 is performed: adjusting the environmental condition setting parameter (Pes) according to the environmental condition corresponding to the minimum value (Pₘᵢₙ) of the penalty function. Please refer to FIG. 5 for example. Assume user A and user D are both in the same room. User A's best value of the thermal sensation indicator (Ib) is −0.2, and user D's best value of the thermal sensation indicator (Ib) is 0.4. The least squares method is taken to obtain a compromised condition for user A and user D. The penalty function (P) is defined as follows:

P=(I_{A}−Ib_{A})²+(I_{D}−Ib_{D})²,

where I_{A} represents the value of the thermal sensation indicator of user A, Ib_{A} represents the best value of the thermal sensation indicator of user A, I_{D} represents the value of the thermal sensation indicator of user D, and Ib_{D} represents the best value of the thermal sensation indicator of user D.

Assume the air temperature in the environment is the controllable parameter. At each sampling moment during sleeping, the penalty function values (P) corresponding to different air temperatures (20˜30 integer degrees Celsius) are calculated. The minimum value (Pₘᵢₙ) of the penalty function and the corresponding air temperature are identified. Then the control module 40 transmits a control signal according to the above air temperature to the air conditioner.

In addition, the sleeping environment control method of the disclosure can also be applied to separating stages for the sleeping time. Each stage of time is not less than the length of sleep cycle, about 60-120 minutes. In an exemplary embodiment, the sleep stage can be separate as: beginning sleep stage (0-2.5 hours), middle sleep stage (2.5-5 hours), and last sleep stage (after 5 hours). For each sleep stage, the sleeping environment control method in the embodiment is respectively and repeatedly performed. As a result, it is helpful to improve the performance of the sleeping environment control system. The reasons are as follows: the user's parameter (Pu), such as the human metabolic rate, as input has a relatively large uncertainty, and the estimated value easily has error. The value of the user's parameter (Pu) would change with the sleep time but can be approximately maintained within a certain specific range for the specific time period. Therefore, if the sleep time is separate into smaller stages, the variances for all the values of the user's parameter in each stage are not changed a lot. In this manner, the error for the estimated values of the user's parameter can be approximately regarded as the constant. In further words, if these errors are approximately constant, then the errors do not significantly reduce the correlation in the regression analysis. The sleeping environment control method of the disclosure can still adjust the environment to the most comfortable condition.

The sleeping environment control system and the method thereof in the disclosure use the physiological status parameter to evaluate the comfortable level during sleeping and use the thermal sensation indicator as the single indicator to evaluate thermal perception. The measured data as collected and the regress analysis are used to establish the correlation function between the physiological status parameter and the thermal sensation indicator, and the influence of sleep cycle causing on the physiological status parameter is excluded. The correlation function is used to find out the best value of the thermal sensation indicator corresponding to the value of the physiological status parameter at the most comfortable condition. And then, by measuring or estimating the un-controllable parameters in the environment condition and the user's parameter, which may influence the value of the thermal sensation indicator, and adjusting the value of the controllable parameter therein, the actual value of the thermal sensation indicator can approach to the best value of the thermal sensation indicator, so as to meet the requirement to have comfortable thermal perception during sleeping. The exemplary embodiment as provided are just for exemplarily describing the features and capability, not for limiting the scope of the disclosure.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A sleeping environment control system, comprising:
a thermal sensation indicator module, adapted to sense a condition of environment to obtain a value of an environmental condition parameter, and generate a value of a thermal sensation indicator according to the value of the environmental condition parameter;
a physiological status module, adapted to sense a physiological status of a user to obtain a value of a physiological status parameter;
an analysis module, comprising a storage unit and a calculating unit, wherein the storage unit receives and stores the value of the thermal sensation indicator from the thermal sensation indicator module and the value of the physiological status parameter from the physiological status module, after a plurality of values of the thermal sensation indicator and a plurality of values of the physiological status parameter are collected, the calculating unit performs a regression analysis to obtain a best value of the thermal sensation indicator according to the plurality of values of the thermal sensation indicator and the plurality of values of the physiological status parameter stored in the storage unit; and
a control module, receiving the best value of the thermal sensation indicator and adjusting a value of an environmental condition setting parameter according to the best value of the thermal sensation indicator.

2. The sleeping environment control system as claimed in claim 1, wherein the environmental condition parameter comprises at least one of air temperature, relative humidity, wind speed, and mean radiant temperature.

3. The sleeping environment control system as claimed in claim 1, wherein the thermal sensation indicator module further receives at least one user's parameter, and generates the value of the thermal sensation indicator according to the value of the environmental condition parameter and a value of the at least one user's parameter.

4. The sleeping environment control system as claimed in claim 3, wherein the at least one user's parameter comprises at least one of the user's fabric thermal resistance and human metabolic rate.

5. The sleeping environment control system as claimed in claim 4, wherein the human metabolic rate is obtained according to at least one of the user's sex, age, height, and weight, a basal metabolic rate formula and a sleep metabolic rate curve.

6. The sleeping environment control system as claimed in claim 1, wherein the physiological status parameter is one of RR parameter, total power (TP), high frequency power (HF) of heart rate variability (HRV), Alpha (α) wave intensity, Beta (β) wave intensity, and Delta (δ) wave intensity of brain waves.

7. The sleeping environment control system as claimed in claim 1, wherein the physiological status module further senses the physiological status of the user to obtain values of an auxiliary physiological signal parameter and transmits the values of the auxiliary physiological signal parameter to the analysis module, the analysis module obtains the user's at least one sleep cycle according to the values of the auxiliary physiological signal parameter, and obtains the plurality of values of the thermal sensation indicator and the plurality of values of the physiological status parameter from the storage unit corresponding to the at least one sleep cycle, and the analysis module further averages the plurality of values of the thermal sensation indicator and the plurality of values of the physiological status parameter to respectively obtain an averaged value of the thermal sensation indicator and an averaged value of the physiological status parameter corresponding to the at least one sleep cycle, and after a plurality of averaged values of the thermal sensation indicator and a plurality of averaged values of the physiological status parameter are collected, performs the regression analysis to obtain the best value of the thermal sensation indicator according to the averaged values of the thermal sensation indicator and the averaged values of the physiological status parameter.

8. The sleeping environment control system as claimed in claim 7, wherein the auxiliary physiological signal parameter is one of RR parameter of heart rate variability, Alpha wave intensity and Delta (δ) wave intensity of brain waves.

9. The sleeping environment control system as claimed in claim 1, wherein the best value of the thermal sensation indicator corresponds to the maximum point or the minimum point of a regression function curve obtained from the regression analysis.

10. The sleeping environment control system as claimed in claim 1, wherein the thermal sensation indicator is one of a comfort index of Pierce two-node model, a thermal sensation vote (TSV) index of KSU two-node model, an index of predicted mean vote (PMV), operative temperature and air temperature.

11. A sleeping environment control method, comprising the steps of:
sensing a condition of environment to obtain a value of an environmental condition parameter, and generating a value of a thermal sensation indicator according to the value of the environmental condition parameter;
sensing a physiological status of a user to obtain a value of a physiological status parameter;
after a plurality of values of the thermal sensation indicator and a plurality of values of the physiological status parameter are collected, performing a regression analysis to obtain a best value of the thermal sensation indicator according to the plurality of values of the thermal sensation indicator and the plurality of values of the physiological status parameter, and
adjusting a value of an environmental condition setting parameter according to the best value of the thermal sensation indicator.

12. The sleeping environment control method as claimed in claim 11, wherein the environmental condition parameter comprises at least one of air temperature, relative humidity, wind speed, and mean radiant temperature.

13. The sleeping environment control method as claimed in claim 11, wherein the step of generating the value of the thermal sensation indicator further comprises:
receiving at least one user's parameter, and
generating the value of the thermal sensation indicator according to the value of the environmental condition parameter and a value of the at least one user's parameter.

14. The sleeping environment control method as claimed in claim 13, wherein the at least one user's parameter comprises at least one of the user's fabric thermal resistance and human metabolic rate.

15. The sleeping environment control method as claimed in claim 14, wherein the human metabolic rate is obtained according to at least one of the user's sex, age, height, and weight, a basal metabolic rate formula and a sleep metabolic rate curve.

16. The sleeping environment control method as claimed in claim 11, wherein the physiological status parameter is one of RR parameter, total power (TP), and high frequency power (HF) of heart rate variability (HRV), Alpha (α) wave intensity, Beta (β) wave intensity and Delta (δ) wave intensity of brain waves.

17. The sleeping environment control method as claimed in claim 11, wherein the method further comprises the steps of:
sensing the physiological status of the user to obtain values of an auxiliary physiological signal parameter;
obtaining the user's at least one sleep cycle according to the values of the auxiliary physiological signal parameter, and obtaining the plurality of values of the thermal sensation indicator and the plurality of values of the physiological status parameter corresponding to the at least one sleep cycle;
averaging the plurality of values of the thermal sensation indicator and the plurality of values of the physiological status parameter to respectively obtain an averaged value of the thermal sensation indicator and an averaged value of the physiological status parameter corresponding to the at least one sleep cycle; and
after a plurality of the averaged values of the thermal sensation indicator and a plurality of the averaged values of the physiological status parameter are collected, performing the regression analysis to obtain the best value of the thermal sensation indicator according to the plurality of the averaged values of the thermal sensation indicator and the plurality of the averaged values of the physiological status parameter.

18. The sleeping environment control method as claimed in claim 17, wherein the auxiliary physiological signal parameter is one of RR parameter of heart rate variability, Alpha wave intensity and Delta (δ) wave intensity of brain waves.

19. The sleeping environment control method as claimed in claim 11, wherein the best value of the thermal sensation indicator corresponds to the maximum point or the minimum point of a regression function curve obtained from the regression analysis.

20. The sleeping environment control method as claimed in claim 11, wherein the thermal sensation indicator is one of a comfort index of Pierce two-node model, a TSV index of KSU two-node model, an index of predicted mean vote (PMV), operative temperature and air temperature.

21. The sleeping environment control method as claimed in claim 11, wherein the step of obtaining the best value of the thermal sensation indicator further comprises:
collecting the plurality of values of the thermal sensation indicator and the plurality of values of the physiological status parameter continuously; and
calculating the best value of the thermal sensation indicator only if the correlation coefficient of the regression analysis is greater than a threshold value.

22. The sleeping environment control method as claimed in claim 11, wherein the value of the thermal sensation indicator is generated from values of a plurality of environmental condition parameters, and the step of adjusting the value of the environmental condition setting parameter further comprises:
selecting a controllable parameter from the plurality of environmental condition parameters as the environmental condition setting parameter, and calculating a value of the controllable parameter causing the value of the thermal sensation indicator to approach the best value of the thermal sensation indicator.

23. The sleeping environment control method as claimed in claim 11 wherein there are a plurality of users and after the plurality of best values of the thermal sensation indicator are obtained, the step of adjusting the environmental condition setting parameter further comprises:
obtaining a minimum value of a penalty function of a least squares method according to the plurality of best values of the thermal sensation indicator; and
adjusting the environmental condition setting parameter according to the environmental condition corresponding to the minimum value of the penalty function.
